(19) United States
(12) Patent Application Publication
(10) Pub. No.: US 2024/0299807 A1
(43) Pub. Date: Sep. 12, 2024

(54) AUTOMATIC EXERCISE ROUTE DETECTION

(71) Applicant: Apple Inc., Cupertino, CA (US)

(72) Inventors: Kenneth M. PESYNA, JR., San Jose, CA (US); Christina SELLE, Los Altos, CA (US)

(21) Appl. No.: 18/118,702

(22) Filed: Mar. 7, 2023

(51) Int. Cl. A63B 24/00; A63B 71/06

(52) U.S. Cl. CPC A63B 24/0062; A63B 71/0622; A63B 2071/0691; A63B 2220/12; A63B 2220/17; A63B 2220/20; A63B 2220/62; A63B 2225/50

(57) ABSTRACT

The subject system may be implemented by at least one processor configured to determine that a location of a user is within a bounding box corresponding to a pre-defined exercise route, receive an indication of a pre-defined segment corresponding to the pre-defined exercise route, and retrieve exercise route data for the pre-defined exercise route. The exercise route data may include pre-determined distance information corresponding to the pre-defined segment of the pre-defined exercise route. The at least one processor may be further configured to receive user location data corresponding to a traversal of the pre-defined segment, generate segment-matched location data by correlating the user location data to the pre-determined distance information corresponding to the pre-defined segment, and provide an exercise metric determined based at least in part on the segment-matched location data.

### TECHNICAL FIELD

The present description generally relates to fitness tracking on electronic devices and, more particularly, to automatic detection of exercise routes to provide more precise fitness tracking.

### BACKGROUND

An electronic device such as a laptop, tablet, smartphone, or a wearable device may include a Global Navigation Satellite System (GNSS) receiver and/or one or more sensors (e.g., an accelerometer, a gyroscope, etc.) that may be used to estimate the position of the electronic device, such as for fitness tracking purposes.

### BRIEF DESCRIPTION OF THE DRAWINGS

Certain features of the subject technology are set forth in the appended claims. However, for the purpose of explanation, several implementations of the subject technology are set forth in the following figures.

FIG. 1 illustrates an example network environment for automatic track detection, in accordance with one or more implementations.

FIG. 2 depicts an example electronic device that may implement the subject methods and systems, in accordance with one or more implementations.

FIG. 3 depicts a geographic region including running tracks within a radius of a user's location, in accordance with one or more implementations.

FIG. 4A depicts running tracks nearby the user, in accordance with one or more implementations.

FIG. 4B depicts a bounding box associated with a running track, in accordance with one or more implementations.

FIG. 5A depicts a confirmation message displayed on a wearable device, in accordance with one or more implementations.

FIG. 5B depicts a lane selection prompt displayed on the wearable device of FIG. 5A, in accordance with one or more implementations.

FIG. 6A depicts map data and track data associated with a running track, in accordance with one or more implementations.

FIG. 6B depicts lane data of the track data of FIG. 6A, in accordance with one or more implementations.

FIG. 7 depicts running data associated with a user running on a running track, in accordance with one or more implementations.

FIG. 8 depicts lane-matched data generated based on the running data, in accordance with or more implementations.

FIG. 9 depicts a flow diagram of an example process for automatic track detection, in accordance with one or more implementations.

FIG. 10 depicts an example electronic system with which aspects of the present disclosure may be implemented, in accordance with one or more implementations.

### DETAILED DESCRIPTION

The detailed description set forth below is intended as a description of various configurations of the subject technology and is not intended to represent the only configurations in which the subject technology can be practiced. The appended drawings are incorporated herein and constitute a part of the detailed description. The detailed description includes specific details for the purpose of providing a thorough understanding of the subject technology. However, the subject technology is not limited to the specific details set forth herein and can be practiced using one or more other implementations. In one or more implementations, structures and components are shown in block diagram form in order to avoid obscuring the concepts of the subject technology.

Users may utilize electronic devices for tracking exercise sessions. Electronic devices may store and/or track location, speed, and time, among other metrics for measuring the effectiveness of an exercise session. Exercise sessions may include, among other things, running track workouts, such as running fixed intervals around a running track. In one approach, a user may manually determine exercise metrics and enter them into the electronic device for storage. In another approach, the user may activate an electronic device configured to utilize one or more sensors to track, and provide for storage, exercise metrics.

Aspects of the subject technology are directed to methods, systems, and computer-readable media for automatically detecting a pre-defined exercise route (e.g., a running track) with an electronic device (e.g., a smartwatch). The electronic device may be configured to utilize one or more sensors to collect exercise metrics as well as determine whether the electronic device is at a location that would be appropriate to collect exercise metrics, such as a running track. In this manner, an electronic device may provide users with a seamless experience from arriving to a running track to beginning an exercise session with minimal configuration of the electronic device for the exercise session. In addition, the electronic device may provide more accurate exercise metrics to the user based on precise map data for each individual running track. For example, precise measurement information corresponding to each lane of a given running track may be downloaded by the electronic device. The electronic device may then use the precise measurement information to refine the position estimation of the electronic device as the user exercises in an identified lane of the running track.

In one or more implementations, the map data may be accessed (e.g., downloaded, retrieved, transferred, etc.) in the background prior to an exercise session so that the user does not need to manually access the map data of the track prior to the exercise session. It should be understood that, although exemplary implementations discussed herein refer to running tracks, the subject technology may be applied to cycling tracks, any other form of an exercise route.

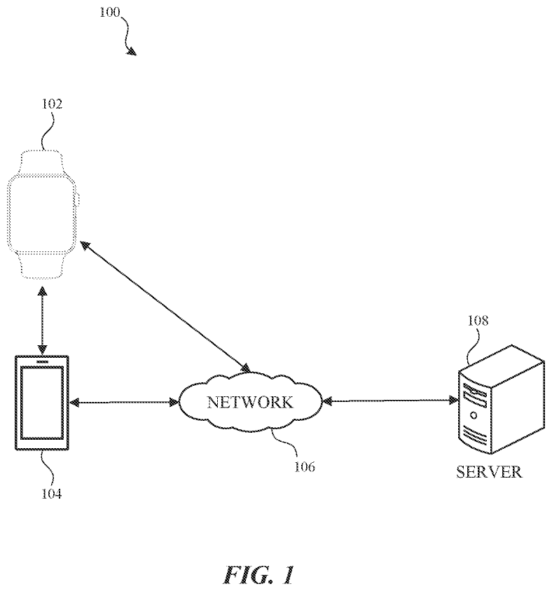

FIG. 1 illustrates an example network environment 100 for automatic track detection, in accordance with one or more implementations. Not all of the depicted components may be used in all implementations, however, and one or more implementations may include additional or different components than those shown in the figure. Variations in the arrangement and type of the components may be made without departing from the spirit or scope of the claims as set forth herein. Additional components, different components, or fewer components may be provided.

The network environment 100 may include an electronic device 102 and one or more servers (e.g., a server 108). The network 106 may communicatively (directly or indirectly) couple the electronic device 102 and the server 108. In one or more implementations, the network 106 may be an interconnected network of devices that may include, or may be communicatively coupled to, the Internet. For explanatory purposes, the network environment 100 is illustrated in FIG. 1 as including the electronic device 102 and the server 108; however, the network environment 100 may include any number of electronic devices and/or any number of servers communicatively coupled to each other directly or via the network 106. Aspects of the subject technology may allow communication of map data at various levels of granularity based on a likelihood (e.g., proximity, locations of interest, and the like) of electronic device 102 and/or electronic device 104 visiting a running track. With the map data, an electronic device (e.g., electronic device 102) may more accurately track exercise sessions.

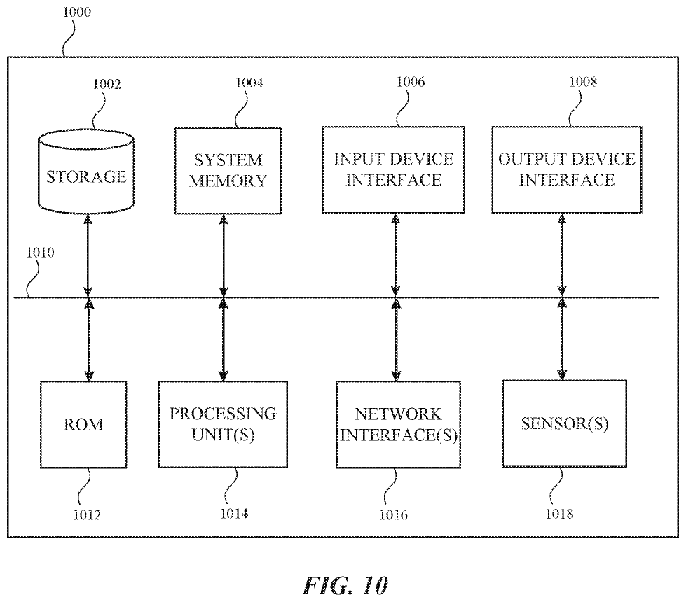

The electronic device 102 may be, for example, a wearable device such as a watch, a band, and the like, a desktop computer, a portable computing device such as a laptop computer, a smartphone, a peripheral device (e.g., a digital camera, headphones), a tablet device, or any other appropriate device that includes, for example, one or more wireless interfaces, such as WLAN radios, cellular radios, Bluetooth radios, Zigbee radios, near field communication (NFC) radios, and/or other wireless radios. In one or more implementations, the electronic device 102 may include a smartwatch configured to track exercise metrics. In FIG. 1, by way of example, the electronic device 102 is depicted as a smartwatch. The electronic device 102 may be, and/or may include all or part of, the electronic system discussed below with respect to FIG. 10. In one or more implementations, the electronic device 102 may include one or more sensors for generating motion data, biometric data, location data, and/or any other data that may be related to an exercise session (e.g., running). In one or more implementations, the electronic device 102 may utilize one or more sensors of the electronic device 104 for generating the motion data, the biometric data, the location data, and/or any other data that may be related to an exercise session. Location sensors may pertain to, for example, positioning technologies such as GNSS positioning, Wi-Fi positioning, cellular phone signal positioning, Bluetooth signal positioning and/or image recognition positioning.

The electronic device 104 may be, for example, a portable computing device such as a laptop computer, a smartphone, a peripheral device (e.g., a digital camera, headphones), a tablet device, a wearable device such as a watch, a band, and the like, any other appropriate device that includes, for example, one or more wireless interfaces, such as WLAN radios, cellular radios, Bluetooth radios, Zigbee radios, NFC radios, and/or other wireless radios. In FIG. 1, by way of example, the electronic device 104 is depicted as a smartphone. The electronic device 104 may be, and/or may include all or part of, the electronic system discussed below with respect to FIG. 10.

In one or more implementations, the electronic device 104 may be configured to be communicatively coupled to the electronic device 102 for the exchange of information such as exercise metrics, map data, location information, and the like. In one or more implementations, the electronic devices 102, 104 may work together to collect, store, and/or analyze sensor data. For example, the electronic device 104 may include a location sensor and communicate location data from the location sensor with the electronic device 102, which may not include a location sensor but rely on the electronic device 104 for determining location data.

In one or more implementations, one or more servers (e.g., the server 108) may store map data of geographic regions at least within a radius of the electronic device 102 and/or electronic device 104. The map data may include locations, bounding boxes, and/or detailed map data of one or more running tracks within the geographic region within the radius. In some variations, the server 108 includes one or more app-specific modules (e.g., plugins) that perform operations for a respective application (e.g., accessing map data).

In one or more implementations, the server 108 may store account information (e.g., user account, user names/handles, or any other account-specific data) associated with the electronic device 102, the electronic device 104, and/or users thereof and/or users associated therewith. In one or more implementations, one or more servers (e.g., the server 108) may provide content (e.g., map content, application content, or any other suitable data) that is to be processed at a participant device (e.g., the electronic device 102 and/or the electronic device 104) by an application or operating system of the participant device.

In one or more implementations, the electronic device 102 may be communicatively coupled to the server 108. The electronic device 102 be configured to communicate with the server 108 to send and/or receive, exercise data, map data, track data, location data, and/or any other information.

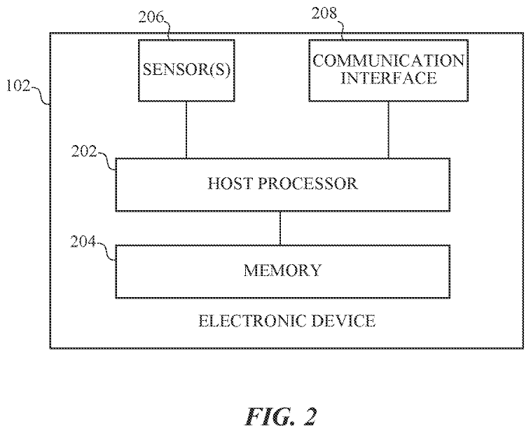

FIG. 2 depicts an example electronic device 102 that may implement the subject methods and systems, in accordance with one or more implementations. For explanatory purposes, FIG. 2 is primarily described herein with reference to the electronic device 102 of FIG. 1. However, this is merely illustrative, and features of the electronic device of FIG. 2 may be implemented in any other electronic device for implementing the subject technology. Not all of the depicted components may be used in all implementations, however, and one or more implementations may include additional or different components than those shown in FIG. 2. Variations in the arrangement and type of the components may be made without departing from the spirit or scope of the claims as set forth herein. Additional components, different components, or fewer components may be provided.

The electronic device 102 may include one or more of a host processor 202, a memory 204, one or more sensor(s) 206, and/or a communication interface 208. The host processor 202 may include suitable logic, circuitry, and/or code that enable processing data and/or controlling operations of the electronic device 102. In this regard, the host processor 202 may be enabled to provide control signals to various other components of the electronic device 102. The host processor 202 may also control transfers of data between various portions of the electronic device 102. The host processor 202 may further implement an operating system or may otherwise execute code to manage operations of the electronic device 102.

The memory 204 may include suitable logic, circuitry, and/or code that enable storage of various types of information such as received data, generated data, code, and/or configuration information. The memory 204 may include volatile memory (e.g., random access memory (RAM)) and/or non-volatile memory (e.g., read-only memory (ROM), flash, and/or magnetic storage). In one or more implementations, the memory 204 may store user location data, track data (e.g., lane information), exercise data (e.g., biometrics), account data, and any other data generated in the course of performing the processes described herein.

The sensor(s) 206 may include one or more motion sensors, biometric sensors, location sensors, and the like. The motion sensors may generate motion data based on the motion of the user (e.g., the user wearing the electronic device 102). The motion data may be used to determine, for example, the number of steps taken, distance traveled, and the like. The biometric sensors may generate biometric data based on, for example, the contact of the sensor with the user. For example, biometric data may include heart rate, respiratory rate, and the like.

The location sensors may generate location data based on a satellite signal, network signal, and the like. The location data may be used to determine, for example, the distance traveled, the proximity to locations of interest (e.g., home or a track), and the like. The location sensors may provide for one or more of GNSS positioning (e.g., via a GNSS receiver configured to receive signals from GNSS satellites), wireless access point positioning (e.g., via a wireless network receiver configured to receive signals from wireless access points), cellular phone signal positioning, Bluetooth signal positioning (e.g., via a Bluetooth receiver), image recognition positioning (e.g., via an image sensor) and/or an INS (e.g., via motion sensors such as an accelerometer and/or gyroscope).

The communication interface 208 may include suitable logic, circuitry, and/or code that enables wired or wireless communication, such as between the electronic device 102 and the server 108. The communication interface 208 may include, for example, one or more of a Bluetooth communication interface, an NFC interface, a Zigbee communication interface, a WLAN communication interface, a USB communication interface, a cellular interface, or generally any communication interface.

In one or more implementations, one or more of the host processor 202, the memory 204, the sensor(s) 206, the communication interface 208, and/or one or more portions thereof may be implemented in software (e.g., subroutines and code), may be implemented in hardware (e.g., an Application Specific Integrated Circuit (ASIC), a Field Programmable Gate Array (FPGA), a Programmable Logic Device (PLD), a controller, a state machine, gated logic, discrete hardware components, or any other suitable devices) and/or a combination of both.

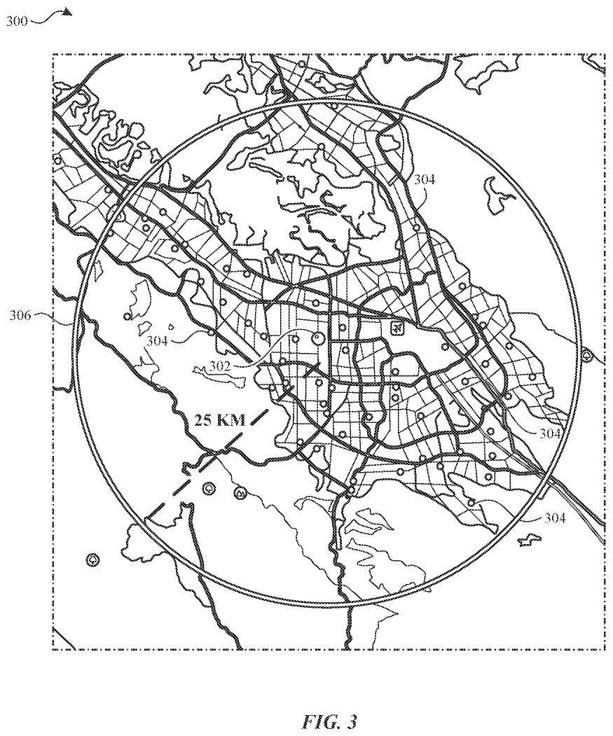

FIG. 3 depicts a geographic region 300 including running tracks 304 within a radius 306 of a user's location 302, in accordance with one or more implementations. Aspects of the subject technology include accessing and storing precise map data of one or more running tracks 304, for example, in advance of the user arriving at the running track.

The electronic device 102 may pre-cache full map data of one or more running tracks 304 in advance of, and/or independent of, the user arriving at any of the running tracks. Pre-caching map data of one or more running tracks 304 (e.g., in advance of and/or independent of the user arriving at any of the running tracks 304) may include downloading the map data and saving the map data to non-volatile storage (e.g., the memory 204) of the electronic device 102. To reduce the amount of storage utilized in pre-caching, the electronic device 102 may download the map data of one or more running tracks 304 within a radius 306 of the user's location 302 (e.g., as determined by the electronic device 102) and/or locations of interest such as the user's home, work, school, or other regularly visited locations.

The radius 306 may be a distance that includes the running tracks 304 that the user is likely to visit, if at all. For example, the user may be likely to visit running tracks 304 within the city of the user's location 302, such as within a 25 kilometer or 15 kilometer radius. The pre-cache radius 306 may be based around the user's location 302 and/or locations of interest. The pre-cache radius 306 may also or instead be around a user's future expected location. For example, when the electronic device 102 is idle and determines the running tracks 304 to pre-cache, the electronic device 102 may identify future expected locations, such as in the user's calendar events, and may then pre-cache the map data of running tracks 304 within a pre-cache radius 306 of the future expected location. In one or more implementations, the electronic device 102 may utilize multiple different radiuses for different locations. For example, the electronic device 102 may utilize a longer radius (e.g., 25 km) around the user's home and work locations but a shorter radius (e.g., 15 km) for other locations that are less likely to be visited, such as a next expected/predicted location (e.g., as determined via calendar events).

In one or more implementations, the pre-caching may occur while the electronic device 102 is idle. The electronic device 102 may be considered idle if the electronic device 102 is unused for a period of time. For example, an electronic device 102 may be considered idle overnight while the electronic device 102 is charging.

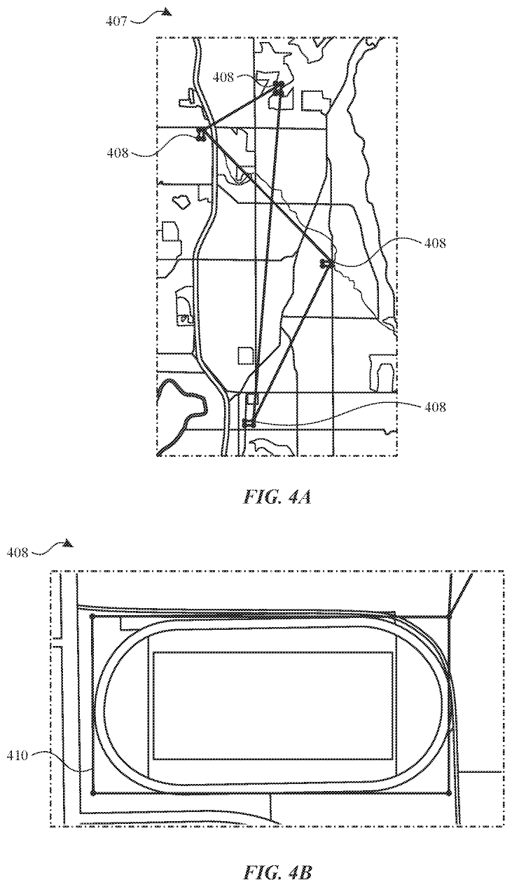

FIG. 4A depicts running tracks 408 nearby the user, in accordance with one or more implementations. Aspects of the subject technology include pre-loading data. As compared to pre-caching, pre-loading data may occur closer to the arrival of the user at a running track 408 and include transferring at least some map data of one or more running tracks 408 from non-volatile memory to volatile memory for use by applications on the electronic device 102.

To reduce the amount of volatile storage utilized in pre-loading, the electronic device 102 may pre-load at least some of the map data of one or more running tracks 408, which may be a subset of the running tracks 304. Pre-loading may occur in advance, and/or independent of, the user arriving at any of the running tracks 408. Pre-loading may include transferring at least some of the map data of one or more running tracks 408 at an area 407 (e.g., as determined by the electronic device 102). The area 407 may be a geographic location around the user that is less than or equal to a geographic region covered by the pre-cache radius 306. For example, if the pre-cache radius 306 is 25 km, a radius of the area 407 may be 5 km. Accordingly, the running tracks 408 may be a subset of the running tracks 304. The area 407 and the pre-loaded data may be periodically refreshed. For example, if the user moves outside of the area 407, the area may be updated to be a 5 km geographic location around the user and the running tracks of the updated area 407 may be pre-loaded.

In one or more implementations, the radius of the area 407 may be based on the density of the running tracks 304 in the geographic region 300 (e.g., the number of pre-cached running tracks 304 in the pre-cache radius 306). For example, if the number of running tracks 304 within a pre-cache radius 306 increases beyond a threshold level of density, the size of the area 407 for pre-loading may decrease so as not to pre-load a number of running tracks 408 beyond a threshold level of running tracks.

FIG. 4B depicts a bounding box 410 associated with a running track 408, in accordance with one or more implementations. A bounding box 410 may be generated by a device storing map data corresponding to a running track 408 (e.g., an electronic device 102 storing map data in in memory 204, a server storing map data in a database, and the like). Map data may include satellite images of a geographic region, which may be analyzed for a subject in an image via a subject recognition algorithm. For example, map data of a region may be analyzed to identify running tracks 408. Part of the analysis may include generating an outline of the identified running tracks 408, where the outline is referred to herein as a bounding box 410. It is contemplated that bounding boxes may be any shape surrounding a running track including, but not limited to, a rectangular shape.

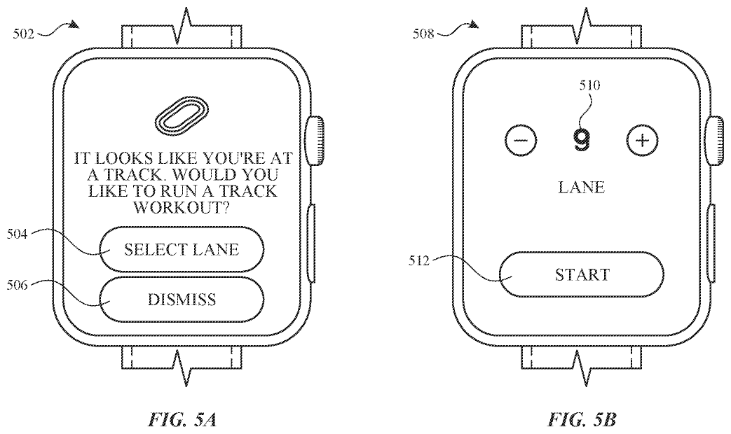

In one or more implementations, the bounding box 410 may operate as a geofence for its associated running track 408. The electronic device 102 may determine that it is within the geofence based on the electronic device's 102 location information. When the electronic device 102 has determined that it is within a threshold proximity to or within the bounding box 410, the electronic device 102 may load the full map data for the running track 408, if it has not done so already (e.g., only bounding boxes were pre-loaded). In one or more implementations, the electronic device 102 may trigger a track detection notification to notify the user that a track has been detected and to solicit a confirmation or other response from the user, as discussed with respect to FIGS. 5A-5B.

The location information may be determined by a satellite signal, such as GPS. Other, less battery-intensive, ways for determining location may be based on a network signal (e.g., a Wi-Fi signal), an NFC signal, an RFID signal, and/or any other signal associated with a geographic location. The electronic device 102 may also or instead utilize a leached location. A leached location may be a location determined by the electronic device 102 for a previous purpose to avoid re-activating the electronic device's 102 location sensor(s) and/or circuitry. For example, the electronic device 102 may determine its location to perform a first task at the current location, and such location may be re-used to perform a second task. A leached location may be utilized if the location was last determined within a time period so as not to be stale. For example, if a determined location was used in performing a first task, the electronic device's 102 location may have changed an hour later so the location may need to be determined for performing a second task.

FIG. 5A depicts a confirmation message 502 displayed on an electronic device 102, in accordance with one or more implementations. A confirmation message 502 may be triggered in response to the electronic device 102 determining that it is in proximity (e.g., within a bounding box or a threshold distance from the bounding box) to a running track. For example, the electronic device 102 may receive location information (e.g., GPS signals) to compare with one or more bounding boxes (e.g., pre-loaded bounding boxes) and determine whether it is within a bounding box. After an electronic device 102 recognizes that it is in proximity to the running track, the electronic device 102 may present a confirmation message 502 notifying the user that a running track has been detected.

The user may confirm the presence of a running track or provide data to the electronic device 102 regarding the running track. For example, the user may press lane selection button 504 to confirm the presence of the running track and to provide a lane (e.g., a pre-defined segment of a pre-defined exercise route) to the electronic device 102 in which the user intends to run. In one or more implementations, the user may ignore the notification. For example, the user may press a dismiss button 506 to remove the notification or may not respond to the notification.

In one or more implementations, the electronic device 102 may check whether the location is nearby the user's home, work, or other location of interest before the electronic device 102 presents the track detection notification to avoid false positive track detections. If the user is near a location of interest, the electronic device 102 may trigger an accurate location determination, rather than leaching the location from a previous, and possibly inaccurate, determined location. An accurate location may be determined via sensors on the electronic device 102 (or any other suitable device), such as GPS or other satellite signals. Additionally or alternatively, the proximity to a track's bounding box for triggering a confirmation message 502 may be reduced so that a user is closer to the track before triggering the confirmation message 502.

FIG. 5B depicts a lane selection prompt 508 displayed on the electronic device 102 of FIG. 5A, in accordance with one or more implementations. If the confirmation message 502 of FIG. 5A solicits a response from the user, the user may interact with the confirmation message 502 (e.g., via the lane selection button 504) to provide information to the electronic device 102, which causes the lane selection prompt 508 to be displayed.

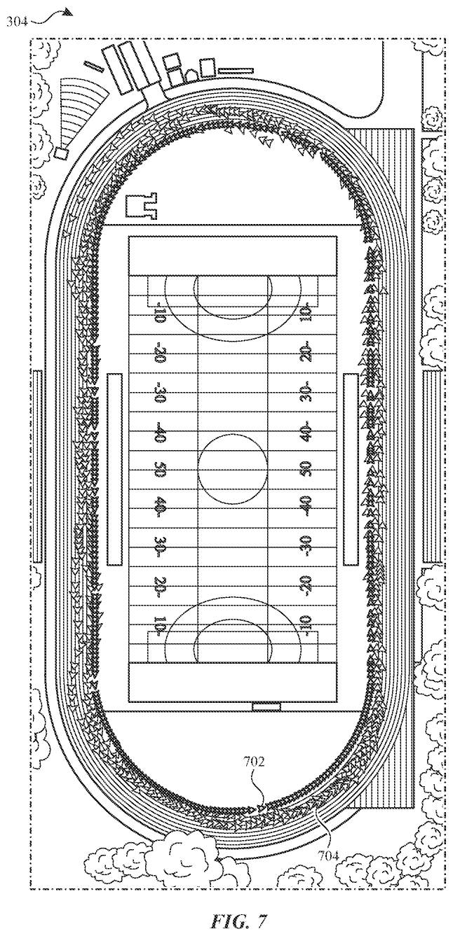

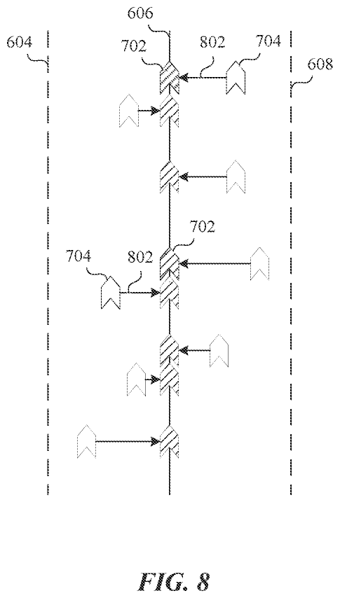

The user may provide the electronic device 102 with the lane in which the user intends to workouts. For example, the user may use the lane selection 510 to indicate a numbered lane of a running track, such as the ninth lane. In one or more implementations, the lane may be automatically determined based on the user's precise location (e.g., via GPS). The lane selection 510 may be utilized to generate lane-matched data, further described below with respect to FIGS. 7-8. When the user selects the start button 512, the electronic device 102 may access map data of the detected track, (e.g., pre-loaded map data as described above with respect to FIGS. 3A-C) and begin generating exercise data (e.g., location data and biometric data). The map data may also or instead be accessed by another electronic device (e.g., from the electronic device 104 and/or a server 108). The lane selection 510 may be used to improve the accuracy of the exercise data in relation to the detected track, as described further below with regard to FIGS. 7-8.

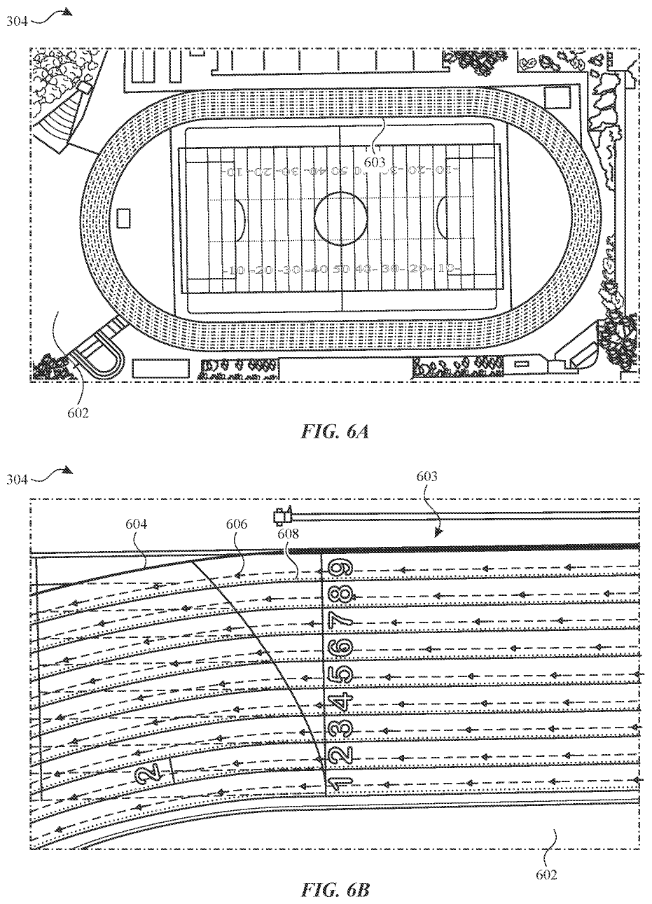

FIG. 6A depicts map data 602 and track data 603 associated with a running track 304, in accordance with one or more implementations. Map data 602 may include data relating to the visual representation of a geographic region including a road map, a satellite view, a terrain map, and the like. For example, the map data 602 is a satellite view of a running track 304. The track data 603 may be derived from the map data 602. For example, a satellite image of a geographic region that includes a running track 304 may be analyzed with a computer vision model to identify standard characteristics of a running track 304, such as shape, lanes, starting lines, lane numbers, and the like. The track data 603 may include one or more data points that define the running track 304 and/or its features, such as lanes and starting lines.

The map data 602 and/or the track data 603 may be accessed (e.g., downloaded, received, retrieved, etc.) by an electronic device 102 from local (e.g., the memory 204) and/or remote storage (e.g., the electronic device 104) when the electronic device 102 nears the running track 304. For example, when the electronic device 102 determines that it is in, or within a threshold distance of, a bounding box of a running track 304, the electronic device 102 may access the map data 602 and/or the track data 603, which was pre-loaded, associated with the running track 304. In one or more implementations, the map data 602 and/or the track data 603 may be accessed after a user confirms that the electronic device 102 is near a running track 304 and/or the user wants to begin a running track 304 workout. For example, a track detection notification (e.g., confirmation message 502) may be triggered in response to the electronic device 102 determining that it is in proximity (e.g., within a bounding box or a threshold distance from the bounding box) to a running track 304, and the user may indicate a lane of the running track 304 and start the workout thereby triggering the electronic device 102 to access the map data 602 and/or the track data 603. In one or more implementations, the track data 603 may be derived from the map data 602 on-the-fly as the map data is accessed.

FIG. 6B depicts lane data of the track data 603 of FIG. 6A, in accordance with one or more implementations. The track data 603 may include one or more data points that define running track 304 lanes and/or their features, such as the lines 604, 608 that define a lane and a path 606. For example, line 604 may be an outer boundary of lane nine and line 608 may be an inner boundary of lane nine, the combination of the two lines 604, 608 define lane nine. A path 606 may be a path that a runner is likely to follow when performing a running track 304 workout. In one or more implementations, the path 606 may be closer to the inner line 608 than the outer line. For example, a runner may typically run closer to the inner line 608 than the outer line 604 because the bends of a running track 304 may lead a runner toward the inner line 608. Accordingly, the electronic device 102 may use the path 606 for lane nine to refine its location estimation while the user is running in lane nine.

FIG. 7 depicts running data 704 associated with a user running on a running track 304, in accordance with one or more implementations. The running track 304 may have associated track data (e.g., track data 603). The track data of running track 304 may be pre-loaded on the electronic device 102 at or before the user initiates a running track workout (e.g., by pressing a start button 512). When the running track workout is initiated, the electronic device 102 may begin generating exercise data, such as location and biometric data. For example, as a user runs laps around a running track 304, the electronic device 102 may track the user's location on the running track and the user's heart rate. The location may be tracked with a location sensor (e.g., a GNSS receiver) on the electronic device 102. However, location sensors are often inaccurate and may measure location data several feet away from the actual location of the location sensor. Inaccuracies in a location sensor may be reflected by inaccuracies in the exercise data, such as the distance traveled, laps run, pace, and any other distance-related metrics.

To compensate for inaccuracies in location data and increase the accuracy of the exercise data, the data points of the location data may be matched to the lane of the running track 304 (e.g., lane-matched data 702), as discussed with respect to FIG. 8.

In one or more implementations, an image of the route the user ran may be overlaid on the map data of the running track 304. The route the user ran may be determined by the lane-matched data 702. For example, the route may be shown as points in the lane-matched data 702 or as a line interpolated through the points in the location data. The route may be colorized based on a metric of the exercise data, such as speed or heart rate. The map data of the running track 304 may include a satellite image of the running track, which may be normalized to level any changes in altitude of the running track. The route (e.g., a line or data points) may be overlaid on the image of the running track so that the user may visualize the workout on the running track 304 for each lap individually, averaged, and/or any other workout-related metric.

FIG. 8 depicts lane-matched data 702 generated based on the running data 704, in accordance with or more implementations. While traversing the running track 304, the location sensor of the electronic device 102 may generate running data 704 and the electronic device 102 may access track data 603 of the running track 304. The track data 603 may include one or more lines 604, 608 that define a lane and a path 606 where the user is likely to run in the lane. The running data 704 may contain inaccuracies that may be inherent in the location sensor. To ameliorate such inaccuracies, lane-matched data 702 may be generated by shifting the running data 704 such that the running data 704 falls along the path 606.

Shifting the running data 704 may include moving the running data 704 in a direction 802 toward the path 606. The running data 704 may be shifted such that the lane-matched data 702 points are substantially the same distance from each other along the path 606 than when they are non-lane-matched (e.g., running data 704). For example, data points of the running data 704 may be shifted onto the path 606 such that the lateral differences between each data point are reduced (e.g., eliminated) but the longitudinal differences between each data point are maintained (e.g., assuming the arrows representing the running data 704 in FIG. 7 are pointing in a northerly direction). The exercise data, such as distance traveled and laps run, may be determined based on the lane-matched data 702.

In one or more implementations, one or more lanes may be normalized such that they are a prescribed (e.g., pre-configured) distance. A running track 304 may have specified measurements to abide by to be utilized for particular events. For example, in an Olympic running track, the first lane may be 400m, the second lane may be 407.67m, and so on. However, not all running tracks 304 have lanes that are the prescribed distance. Accordingly, to track exercise data based on the expected length of the lane, track data 603 corresponding to the lane (which includes the path 606) may be lengthened or shortened so that it conforms to the prescribed length. When generating the lane-matched data 702, the running data 704 points may be shifted toward the path 606 and shifted toward the ends of the path 606 (e.g., a start and/or finish line) such that the distance between running data 704 points are shifted further from or closer to each other in an amount commensurate to the amount that the lane was lengthened or shortened.

Normalization of the running data 704 may occur in real-time or periodically. The adjustment may occur on lap-by-lap periods or portions thereof, such as every 400m or every 200m. The normalization may also or instead occur on epoch-by-epoch periods, such as every 10 seconds. The exercise data, such as distance traveled and laps run, may be determined based on the normalized lane-matched data 702.

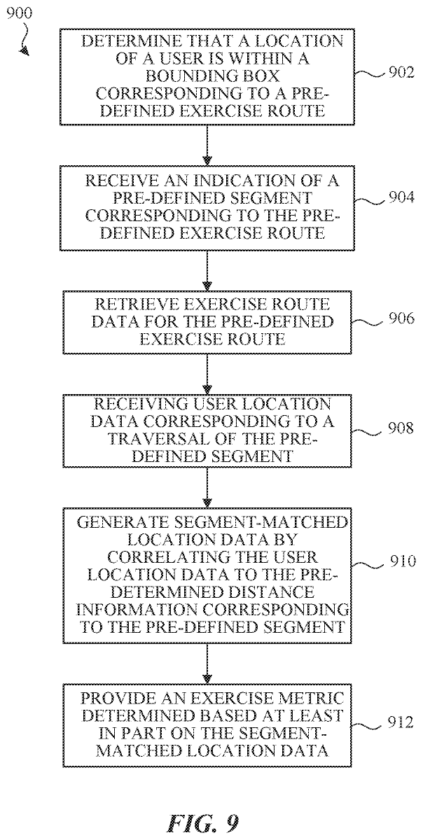

FIG. 9 depicts a flow diagram of an example process 900 for automatic running track detection, in accordance with one or more implementations. For explanatory purposes, the process 900 is primarily described herein with reference to the electronic device 102, the electronic device 104, and the server 108 of FIG. 1. However, the process 900 is not limited to the electronic device 102, the electronic device 104, or the server 108, and one or more blocks of the process 900 may be performed by one or more other components of the server 108, and/or by other suitable devices. Further, for explanatory purposes, the blocks of the process 900 are described herein as occurring sequentially or linearly. However, multiple blocks of the process 900 may occur in parallel. In addition, the blocks of the process 900 need not be performed in the order shown and/or one or more blocks of the process 900 need not be performed and/or can be replaced by other operations.

At block 902, an electronic device 102 may determine whether a user's location is within a bounding box corresponding to a pre-defined exercise route (e.g., running track 304). A pre-defined exercise route may include a running track, cycling track, trail, road, sidewalk, user-defined route, or any other defined route. The exercise route and/or a representation of the exercise route may be stored on the electronic device 102, or on another electronic device (e.g., the electronic device 104 or server 108), and pre-cached/pre-loaded on the electronic device 102. One representation of the exercise route may be a bounding box (e.g., the bounding box 410), which may be a shape that surrounds the geographic location of the exercise route. The bounding box may be generated based on the pre-defined exercise route. For example, the server 108 may analyze map data with a machine learning model trained to identify exercise routes in the shape of a running track, where identifying the exercise routes includes isolating the exercise routes with a bounding box.

One or more bounding boxes associated with one or more exercise routes may be pre-cached on the electronic device 102. The electronic device 102 may periodically access (e.g., query, retrieve, load, receive) a repository of bounding boxes to locally store one or more bounding boxes nearby the electronic device 102 and/or one or more locations of interest. Locations of interest may include the current location of the user and/or expected future locations of the user. For example, the electronic device 102 may pre-cache (e.g., store in non-volatile memory) one or more bounding boxes within a pre-defined distance of a user, such as within a 25 km radius of the user. The electronic device 102 may also pre-load (store in volatile memory) one or more bounding boxes within a pre-defined of one or more locations of interest, such as within a 2 km radius of locations indicated in the user's calendar events for the following day.

To determine that a user is within a bounding box, the electronic device 102 may determine its location. The location may be based at least in part on a satellite signal (e.g., a GPS signal) and/or a network signal (e.g., a Wi-Fi signal). The location may be compared with the locations covered by the bounding boxes accessed (e.g., stored) by the electronic device 102. When the location is within a geographic area covered by a bounding box, a notification (e.g., confirmation message 502) may be provided to the user for the user to confirm that he is at or near the pre-defined exercise route. The user may provide a confirmation (e.g., via a lane selection button 504 on the notification) that the user is at the pre-defined exercise route associated with the bounding box.

At block 904, the electronic device 102 may receive an indication of a pre-defined segment corresponding to the pre-defined exercise route. Pre-defined exercise routes may include one or more pre-defined segments. Segments may be portions of the exercise route that divide the exercise route into sections. For example, if the exercise route is a running track 304, the segments may be lanes of the running track 304. After the electronic device 102 has determined that it is at an exercise route, the electronic device 102 may determine the segment of the exercise route to be utilized by the user. In one or more implementations, the electronic device 102 may generate a segment selection message (e.g., a lane selection prompt 508). The segment selection message may present one or more available segments for the user to select. The user may indicate a selection by interacting with the segment selection message by scrolling to the selection, selecting the selection, speaking the selection, and any other form of selecting a segment.

At block 906, the electronic device 102 may retrieve exercise route data for the pre-defined exercise route. The exercise route data may include the detailed information about the exercise route, such as pre-determined distance information, pre-defined exercise route, pre-configured distance. For example, if the exercise route is a running track 304, the segment may be a lane (e.g., defined by lines 604, 608), the pre-determined distance information may be a path 606 having a pre-configured distance of 400m at the innermost lane. The exercise route data may be retrieved from local and/or external sources. For example, the exercise route data may be retrieved from a non-volatile and/or volatile memory on the electronic device 102 or from a database (e.g., at the server 108).

At block 908, the electronic device 102 may receive user location data corresponding to a traversal of the pre-defined segment. The electronic device 102 may receive the user location data from a local sensor and/or a sensor from another device (e.g., the electronic device 104), one or both of which may accompany the user on the exercise route. User location data may be collected as the user traverses the exercise route, such as a segment of the exercise route.

At block 910, the electronic device 102 may generate segment-matched location data. The electronic device 102 may correlate the user location data from block 908 to the pre-determined distance information from block 906. Correlating the user location data to the distance information may include adjusting one or more data points of the user location to the distance information. For example, the location data (e.g., running data 704) may be adjusted to align with the distance information of a segment (e.g., a path 606 of a running track's lane). In one or more implementations, path information corresponding to the lane selected by the user may be provided to a Kalman filter used by the electronic device 102 for position estimation.

In one or more implementations, adjusting may include shifting a data point toward the distance information (e.g., the path 606), as described with regard to FIG. 7 above. The one or more data points may be adjusted in a direction most direct to the distance information. The one or more data points may be adjusted such that the data points of the segment-matched location data (e.g., lane-matched data 702) are substantially the same distance from each other along the distance information than when they are non-segment-matched (e.g., running data 704). For example, data points of the user location data may be shifted onto the location data such that the lateral differences between each data point of the user location data are reduced (e.g., eliminated) but the longitudinal differences between each data point of the user location are maintained.

In one or more implementations, the pre-determined distance information of the segment from block 904 may be adjusted to match a pre-configured distance so that the segment-matched location data is normalized to the pre-configured distance when generating segment-matched location data, as described with regard to FIG. 8 above.

At block 912, the electronic device 102 may provide exercise metric(s) determined based at least in part on the segment-matched location data. The segment-matched location data may be used to determine a lap count, a lap duration, a lap distance (e.g., a portion of a lap run), and/or a total distance (e.g., total distance run). For example, a lap count may be determined by counting the number of a start line is crossed, where a start line is the first point of location data, and a lap duration may be the amount of time it takes for a traversal of the segment to cross the start line.

As described above, one aspect of the present technology is the gathering and use of data available from specific and legitimate sources for file sharing. The present disclosure contemplates that in some instances, this gathered data may include personal information data that uniquely identifies or can be used to identify a specific person. Such personal information data can include demographic data, location-based data, online identifiers, telephone numbers, email addresses, home addresses, images, videos, audio data, data or records relating to a user's health or level of fitness (e.g., vital signs measurements, medication information, exercise information), date of birth, or any other personal information.

The present disclosure recognizes that the use of such personal information data, in the present technology, can be used to the benefit of users. For example, personal information data can be used for file sharing. Accordingly, the use of such personal information data may facilitate transactions (e.g., online transactions). Further, other uses for personal information data that benefit the user are also contemplated by the present disclosure. For instance, health and fitness data may be used, in accordance with the user's preferences to provide insights into their general wellness or may be used as positive feedback to individuals using technology to pursue wellness goals.

The present disclosure contemplates that those entities responsible for the collection, analysis, disclosure, transfer, storage, or other use of such personal information data will comply with well-established privacy policies and/or privacy practices. In particular, such entities would be expected to implement and consistently apply privacy practices that are generally recognized as meeting or exceeding industry or governmental requirements for maintaining the privacy of users. Such information regarding the use of personal data should be prominently and easily accessible by users and should be updated as the collection and/or use of data changes. Personal information from users should be collected for legitimate uses only. Further, such collection/sharing should occur only after receiving the consent of the users or other legitimate basis specified in applicable law. Additionally, such entities should consider taking any needed steps for safeguarding and securing access to such personal information data and ensuring that others with access to the personal information data adhere to their privacy policies and procedures. Further, such entities can subject themselves to evaluation by third parties to certify their adherence to widely accepted privacy policies and practices. In addition, policies and practices should be adapted for the particular types of personal information data being collected and/or accessed and adapted to applicable laws and standards, including jurisdiction-specific considerations which may serve to impose a higher standard. For instance, in the US, collection of or access to certain health data may be governed by federal and/or state laws, such as the Health Insurance Portability and Accountability Act (HIPAA); whereas health data in other countries may be subject to other regulations and policies and should be handled accordingly.

Despite the foregoing, the present disclosure also contemplates implementations in which users selectively block the use of, or access to, personal information data. That is, the present disclosure contemplates that hardware and/or software elements can be provided to prevent or block access to such personal information data. For example, in the case of file sharing, the present technology can be configured to allow users to select to “opt-in” or “opt-out” of participation in the collection of personal information data during registration for services or anytime thereafter. In addition to providing “opt-in” and “opt-out” options, the present disclosure contemplates providing notifications relating to the access or use of personal information. For instance, a user may be notified upon downloading an app that their personal information data will be accessed and then reminded again just before personal information data is accessed by the app.

Moreover, it is the intent of the present disclosure that personal information data should be managed and handled in a way to minimize risks of unintentional or unauthorized access or use. Risk can be minimized by limiting the collection of data and deleting data once it is no longer needed. In addition, and when applicable, including in certain health-related applications, data de-identification can be used to protect a user's privacy. De-identification may be facilitated, when appropriate, by removing identifiers, controlling the amount or specificity of data stored (e.g., collecting location data at city level rather than at an address level), controlling how data is stored (e.g., aggregating data across users), and/or other methods such as differential privacy.

Therefore, although the present disclosure broadly covers use of personal information data to implement one or more various disclosed implementations, the present disclosure also contemplates that the various implementations can also be implemented without the need for accessing such personal information data. That is, the various implementations of the present technology are not rendered inoperable due to the lack of all or a portion of such personal information data.

FIG. 10 depicts an example electronic system 1000 with which aspects of the present disclosure may be implemented, in accordance with one or more implementations. The electronic system 1000 can be, and/or can be a part of, any electronic device for generating the features and processes described in reference to FIGS. 1-9, including but not limited to a laptop computer, tablet computer, smartphone, and wearable device (e.g., smartwatch, fitness band). The electronic system 1000 may include various types of computer-readable media and interfaces for various other types of computer-readable media. The electronic system 1000 includes one or more processing unit(s) 1014, a persistent storage device 1002, a system memory 1004 (and/or buffer), an input device interface 1006, an output device interface 1008, a bus 1010, a ROM 1012, one or more processing unit(s) 1014, one or more network interface(s) 1016, one or more sensors 1018, and/or subsets and variations thereof.

The bus 1010 collectively represents all system, peripheral, and chipset buses that communicatively connect the numerous internal devices of the electronic system 1000. In one or more implementations, the bus 1010 communicatively connects the one or more processing unit(s) 1014 with the ROM 1012, the system memory 1004, and the persistent storage device 1002. From these various memory units, the one or more processing unit(s) 1014 retrieves instructions to execute and data to process in order to execute the processes of the subject disclosure. The one or more processing unit(s) 1014 can be a single processor or a multi-core processor in different implementations.

The ROM 1012 stores static data and instructions that are needed by the one or more processing unit(s) 1014 and other modules of the electronic system 1000. The persistent storage device 1002, on the other hand, may be a read-and-write memory device. The persistent storage device 1002 may be a non-volatile memory unit that stores instructions and data even when the electronic system 1000 is off. In one or more implementations, a mass-storage device (such as a magnetic or optical disk and its corresponding disk drive) may be used as the persistent storage device 1002.

In one or more implementations, a removable storage device (such as a floppy disk, flash drive, and its corresponding disk drive) may be used as the persistent storage device 1002. Like the persistent storage device 1002, the system memory 1004 may be a read-and-write memory device. However, unlike the persistent storage device 1002, the system memory 1004 may be a volatile read-and-write memory, such as RAM. The system memory 1004 may store any of the instructions and data that one or more processing unit(s) 1014 may need at runtime. In one or more implementations, the processes of the subject disclosure are stored in the system memory 1004, the persistent storage device 1002, and/or the ROM 1012. From these various memory units, the one or more processing unit(s) 1014 retrieves instructions to execute and data to process in order to execute the processes of one or more implementations.

The bus 1010 also connects to the input device interfaces 1006 and output device interfaces 1008. The input device interface 1006 enables a user to communicate information and select commands to the electronic system 1000. Input devices that may be used with the input device interface 1006 may include, for example, alphanumeric keyboards, touch screens, and pointing devices. The output device interface 1008 may enable the electronic system 1000 to communicate information to users. For example, the output device interface 1008 may provide the display of images generated by electronic system 1000. Output devices that may be used with the output device interface 1008 may include, for example, printers and display devices, such as a liquid crystal display (LCD), a light emitting diode (LED) display, an organic light emitting diode (OLED) display, a flexible display, a flat panel display, a solid-state display, a projector, or any other device for outputting information.

One or more implementations may include devices that function as both input and output devices, such as a touchscreen. In these implementations, feedback provided to the user can be any form of sensory feedback, such as visual feedback, auditory feedback, or tactile feedback; and input from the user can be received in any form, including acoustic, speech, or tactile input.

The bus 1010 also connects to sensor(s) 1018. The sensor(s) 1018 may include a location sensor, which may be used in determining device position based on positioning technology. For example, the location sensor may provide for one or more of GNSS positioning, wireless access point positioning, cellular phone signal positioning, Bluetooth signal positioning, image recognition positioning, and/or an INS (e.g., via motion sensors such as an accelerometer and/or gyroscope). In one or more implementations, the sensor(s) 1018 may be utilized to detect movement, travel and orientation of the electronic system 1000. For example, the sensor(s) may include an accelerometer, a rate gyroscope, and/or other motion-based sensor(s). The sensor(s) 1018 may include one or more biometric sensors for determining the exercise data of a user such as heart rate, respiratory rate, and any other biometric data.

Finally, as shown in FIG. 10, the bus 1010 also couples the electronic system 1000 to one or more networks and/or to one or more network nodes through the one or more network interface(s) 1016. In this manner, the electronic system 1000 can be a part of a network of computers (such as a local area network, a wide area network, an Intranet, or a network of networks, such as the Internet). Any or all components of the electronic system 1000 can be used in conjunction with the subject disclosure.

Implementations within the scope of the present disclosure can be partially or entirely realized using a tangible computer-readable storage medium (or multiple tangible computer-readable storage media of one or more types) encoding one or more computer-readable instructions. The tangible computer-readable storage medium also can be non-transitory in nature.

The computer-readable storage medium can be any storage medium that can be read, written, or otherwise accessed by a general purpose or special purpose computing device, including any processing electronics and/or processing circuitry capable of executing instructions. For example, without limitation, the computer-readable medium can include any volatile semiconductor memory, such as RAM, DRAM, SRAM, T-RAM, Z-RAM, and TTRAM. The computer-readable medium also can include any non-volatile semiconductor memory, such as ROM, PROM, EPROM, EEPROM, NVRAM, flash, nvSRAM, FeRAM, FeTRAM, MRAM, PRAM, CBRAM, SONOS, RRAM, NRAM, racetrack memory, FJG, and Millipede memory.

Further, the computer-readable storage medium can include any non-semiconductor memory, such as optical disk storage, magnetic disk storage, magnetic tape, other magnetic storage devices, or any other medium capable of storing one or more instructions. In one or more implementations, the tangible computer-readable storage medium can be directly coupled to a computing device, while in other implementations, the tangible computer-readable storage medium can be indirectly coupled to a computing device, e.g., via one or more wired connections, one or more wireless connections, or any combination thereof.

Instructions can be directly executable or can be used to develop executable instructions. For example, instructions can be realized as executable or non-executable machine code or as instructions in a high-level language that can be compiled to produce executable or non-executable machine code. Further, instructions also can be realized as or can include data. Computer-executable instructions also can be organized in any format, including routines, subroutines, programs, data structures, objects, modules, applications, applets, functions, etc. As recognized by those of skill in the art, details including, but not limited to, the number, structure, sequence, and organization of instructions can vary significantly without varying the underlying logic, function, processing, and output.

While the above discussion primarily refers to microprocessors or multi-core processors that execute software, one or more implementations are performed by one or more integrated circuits, such as ASICs or FPGAs. In one or more implementations, such integrated circuits execute instructions that are stored on the circuit itself.

Those of skill in the art would appreciate that the various illustrative blocks, modules, elements, components, methods, and algorithms described herein may be implemented as electronic hardware, computer software, or combinations of both. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. Skilled artisans may implement the described functionality in varying ways for each particular application. Various components and blocks may be arranged differently (e.g., arranged in a different order, or partitioned in a different way), all without departing from the scope of the subject technology.

It is understood that any specific order or hierarchy of blocks in the processes disclosed is an illustration of example approaches. Based upon design preferences, it is understood that the specific order or hierarchy of blocks in the processes may be rearranged, or that all illustrated blocks be performed. Any of the blocks may be performed simultaneously. In one or more implementations, multitasking and parallel processing may be advantageous. Moreover, the separation of various system components in the implementations described above should not be understood as requiring such separation in all implementations, and it should be understood that the described program components and systems can generally be integrated together in a single software product or packaged into multiple software products.

As used in this specification and any claims of this application, the terms “base station,” “receiver,” “computer,” “server,” “processor,” and “memory” all refer to electronic or other technological devices. These terms exclude people or groups of people. For the purposes of the specification, the terms “display” or “displaying” means displaying on an electronic device.

As used herein, the phrase “at least one of” preceding a series of items, with the term “and” or “or” to separate any of the items, modifies the list as a whole, rather than each member of the list (i.e., each item). The phrase “at least one of” does not require selection of at least one of each item listed; rather, the phrase allows a meaning that includes at least one of any one of the items, and/or at least one of any combination of the items, and/or at least one of each of the items. By way of example, the phrases “at least one of A, B, and C” or “at least one of A, B, or C” each refers to only A, only B, or only C; any combination of A, B, and C; and/or at least one of each of A, B, and C.

The predicate words “configured to,” “operable to,” and “programmed to” do not imply any particular tangible or intangible modification of a subject, but, rather, are intended to be used interchangeably. In one or more implementations, a processor configured to monitor and control an operation or a component may also mean the processor being programmed to monitor and control the operation or the processor being operable to monitor and control the operation. Likewise, a processor configured to execute code can be construed as a processor programmed to execute code or operable to execute code.

Phrases such as an aspect, the aspect, another aspect, some aspects, one or more aspects, an implementation, the implementation, another implementation, one or more implementations, one or more implementations, an embodiment, the embodiment, another embodiment, one or more implementations, one or more implementations, a configuration, the configuration, another configuration, some configurations, one or more configurations, the subject technology, the disclosure, the present disclosure, other variations thereof and alike are for convenience and do not imply that a disclosure relating to such phrase(s) is essential to the subject technology or that such disclosure applies to all configurations of the subject technology. A disclosure relating to such phrase(s) may apply to all configurations, or one or more configurations. A disclosure relating to such phrase(s) may provide one or more examples. A phrase such as an aspect or some aspects may refer to one or more aspects and vice versa, and this applies similarly to other foregoing phrases.

The word “exemplary” is used herein to mean “serving as an example, instance, or illustration.” Any implementation described herein as “exemplary” or as an “example” is not necessarily to be construed as preferred or advantageous over other implementations. Furthermore, to the extent that the term “include,” “have,” or the like is used in the description or the claims, such term is intended to be inclusive in a manner similar to the term “comprise” as “comprise” is interpreted when employed as a transitional word in a claim.

All structural and functional equivalents to the elements of the various aspects described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.”

The previous description is provided to enable any person skilled in the art to practice the various aspects described herein. Various modifications to these aspects will be readily apparent to those skilled in the art, and the generic principles defined herein may be applied to other aspects. Thus, the claims are not intended to be limited to the aspects shown herein but are to be accorded the full scope consistent with the language claims, wherein reference to an element in the singular is not intended to mean “one and only one” unless specifically so stated, but rather “one or more.” Unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the subject disclosure.

What is claimed is:

1. A method comprising:
determining that a location of a user is within a bounding box corresponding to a pre-defined exercise route;
receiving an indication of a pre-defined segment corresponding to the pre-defined exercise route;
retrieving exercise route data for the pre-defined exercise route, wherein the exercise route data comprises pre-determined distance information corresponding to the pre-defined segment of the pre-defined exercise route;
receiving user location data corresponding to a traversal of the pre-defined segment;
generating segment-matched location data by correlating the user location data to the pre-determined distance information corresponding to the pre-defined segment; and
providing an exercise metric determined based at least in part on the segment-matched location data.

2. The method of claim 1, further comprising, prior to determining that the location of the user is within the bounding box:
accessing a location of interest of the user; and
accessing bounding boxes corresponding to pre-defined exercise routes within a radius of the location of interest.

3. The method of claim 1, wherein determining that the location of the user is within the bounding box comprises:
providing a confirmation message that the location of the user is at the pre-defined exercise route in response to determining that the location of the user is within the bounding box; and
receiving, from the user and responsive to the confirmation message, a confirmation indicating that the location of the user is at the pre-defined exercise route associated with the bounding box based on the confirmation message.

4. The method of claim 1, wherein determining that the location of the user is within the bounding box is based at least in part on at least one of a satellite signal or a network signal.

5. The method of claim 1, wherein the receiving of the indication of the pre-defined segment comprises:
generating a segment selection message indicating one or more pre-defined segments of the pre-defined exercise route; and
receiving a selection of the pre-defined segment from the one or more pre-defined segments in response to the segment selection message.

6. The method of claim 1, further comprising:
adjusting the pre-determined distance information corresponding to the pre-defined segment to match a pre-configured distance.

7. The method of claim 1, wherein the pre-defined exercise route is a running track and the pre-defined segment is a lane.

8. The method of claim 7, wherein the exercise metric comprises one or more of a lap count, a lap duration, and a lap distance.

9. The method of claim 1, wherein the exercise route data is derived from a map data associated with the pre-defined exercise route.

10. A device comprising:
a memory; and
a processor configured to:
determine that a location of a user is within a bounding box corresponding to a pre-defined exercise route;
receive an indication of a pre-defined segment corresponding to the pre-defined exercise route;
retrieve exercise route data for the pre-defined exercise route, wherein the exercise route data comprises pre-determined distance information corresponding to the pre-defined segment of the pre-defined exercise route;
receive user location data corresponding to a traversal of the pre-defined segment;
generate segment-matched location data by correlating the user location data to the pre-determined distance information corresponding to the pre-defined segment; and
provide an exercise metric determined based at least in part on the segment-matched location data.

11. The device of claim 10, wherein the processor is further configured to, prior to a determination that the location of the user is within the bounding box:
access a location of interest of the user; and
access bounding boxes corresponding to pre-defined exercise routes within a radius of the location of interest.

12. The device of claim 10, wherein the processor is configured to determine that the location of the user is within the bounding box by:
providing a confirmation message that the location of the user is at the pre-defined exercise route in response to determining that the location of the user is within the bounding box; and
receiving, from the user and responsive to the confirmation message, a confirmation indicating that the location of the user is at the pre-defined exercise route associated with the bounding box based on the confirmation message.

13. The device of claim 10, wherein the processor is configured to determine that the location of the user is within the bounding box based at least in part on at least one of a satellite signal or a network signal.

14. The device of claim 10, wherein the processor is further configured to:
adjust the pre-determined distance information corresponding to the pre-defined segment to match a pre-configured distance.

15. The device of claim 10, wherein the pre-defined exercise route is a running track and the pre-defined segment is a lane.

16. The device of claim 15, wherein the exercise metric comprises one or more of a lap count, a lap duration, and a lap distance.

17. The device of claim 10, wherein the exercise route data is derived from a map data associated with the pre-defined exercise route.

18. A non-transitory computer-readable medium comprising computer-readable instructions that, when executed by a processor, cause the processor to perform one or more operations comprising:
determining that a location of a user is within a bounding box corresponding to a pre-defined exercise route;
receiving an indication of a pre-defined segment corresponding to the pre-defined exercise route;
retrieving exercise route data for the pre-defined exercise route, wherein the exercise route data comprises pre-determined distance information corresponding to the pre-defined segment of the pre-defined exercise route;
receiving user location data corresponding to a traversal of the pre-defined segment;
generating segment-matched location data by correlating the user location data to the pre-determined distance information corresponding to the pre-defined segment; and
providing an exercise metric determined based at least in part on the segment-matched location data.

19. The non-transitory computer-readable medium of claim 18, wherein the operations further comprise, prior to determining that the location of the user is within the bounding box:
accessing a location of interest of the user; and
accessing bounding boxes corresponding to pre-defined exercise routes within a radius of the location of interest.

20. The non-transitory computer-readable medium of claim 18, wherein determining that the location of the user is within the bounding box comprises:
providing a confirmation message that the location of the user is at the pre-defined exercise route in response to determining that the location of the user is within the bounding box; and
receiving, from the user and responsive to the confirmation message, a confirmation indicating that the location of the user is at the pre-defined exercise route associated with the bounding box based on the confirmation message.
